(19) United States
(12) Patent Application Publication
(10) Pub. No.: US 2026/0166200 A1
(43) Pub. Date: Jun. 18, 2026

(54) BONE TISSUE GROWTH SUPPORTING DEVICE

(71) Applicant: Xerapeutics AB, Uppsala (SE)

(72) Inventor: Håkan ENGQVIST, Uppsala (SE)

(21) Appl. No.: 19/424,620

(22) Filed: Dec. 18, 2025

Related U.S. Application Data
(60) Provisional application No. 63/735,576, filed on Dec. 18, 2024.

(51) Int. Cl. A61L 27/38; A61L 27/24; A61L 27/30; A61L 27/36; A61L 27/56; A61L 27/58; C08L 89/04

(52) U.S. Cl. CPC A61L 27/3847; A61L 27/24; A61L 27/306; A61L 27/3608; A61L 27/56; A61L 27/58; C08L 89/04; A61L 2430/02

(57) ABSTRACT

A device for supporting bone tissue growth at a bone defect comprises a deformable semipermeable pouch made of a bioabsorbable material and bone graft granules enclosed by the deformable semipermeable pouch. The bone graft granules comprise a calcium salt. The deformable semipermeable pouch preferably comprises at least one through hole arranged and dimensioned for reception of a fastening screw configured to screw into a bone and attach the deformable semipermeable pouch at the bone.

### TECHNICAL FIELD

The present invention generally relates to devices for supporting bone tissue growth, and in particular such devices designed to be used in connection with non-confined bone defects.

### BACKGROUND

Bone reconstruction is a challenge and complex process, involving well-defined biochemical mechanisms, which are essential for the mobilization of cell populations and crosstalk between them. The intrinsic capacity of bone tissue to regenerate can be challenging in complex clinical cases due to excessive damage caused by, for example, trauma, tumors, infection, surgery or due to various skeletal diseases or disorders. An improved understanding of bone tissue and the cellular and biochemical mechanisms involved in bone healing has allowed the emergence of numerous bone grafting strategies.

These strategies involve, but are not limited to, supplementing the bone healing capacity with bone tissue taken from the patient (autogenous graft or allograft) or another patient (allogenous graft or allograft), with the use of synthetic bone grafts as well as introducing various growth factors.

Autogenous grafts remain the gold standard for bone grafting as it naturally contains the right amount of growth factors and carries no risks for rejection or transferal of disease as allografts. However, it still carries an increased risk of infection and donor site morbidity. Furthermore, the patient's overall health may prohibit its use, or the autograft may be resorbed too quickly. Allografts require treatment to reduce immunogenicity and are marred by the potential risks of disease transmission from the donor to the recipient. As a results, a variety of synthetic bone graft substitutes have been developed, out of which ceramic-based grafts are the most used today.

Synthetic bone grafts can be in various forms, including powders or granules, putty or paste, a block or other pre-formed three-dimensional (3D) shape and injectable bone cements, each having pros and cons. Powders or granules are widely used due to their simplicity and versatility but are generally limited to so-called confined bone defects as the powder particles or granules may otherwise migrate out of the bone defects. A putty or paste is pre-mixed with binding agents and typically injected to allow for minimally invasive surgery. Such synthetic bone grafts may also have migration problems and are therefore mainly limited to confined bone defects. Blocks or other pre-formed 3D shapes require precise surgical fitting at a bone defect or complex operations if manufactured to be customized to specifically match a patient-specific bone defect. Finally, injectable bone cements require additional mixing operations at site to form a flowable paste that hardens in situ. The injectable bone cements have similar migration problems as powders or granules and putties or pastes and are thereby mainly used in confined bone defects.

There is, thus, a need for a device for supporting bone tissue growth and that can be used also for non-confined bone defects with at least a reduced risk of migration of the synthetic bone grafts.

### SUMMARY

It is an objective to provide a device for supporting bone tissue growth and that can be used for non-confined bone defects.

This and other objectives are met by embodiments of the present invention.

The present invention is defined in the independent claims. Further embodiments of the invention are defined in the dependent claims.

An aspect of the invention relates to a device for supporting bone tissue growth at a bone defect. The device comprises a deformable semipermeable pouch made of a bioabsorbable material, and bone graft granules enclosed by the deformable semipermeable pouch. The bone graft granules comprise a calcium salt. The deformable semipermeable pouch comprises at least one through hole arranged and dimensioned for reception of a fastening screw configured to screw into a bone and attach the deformable semipermeable pouch at the bone.

Another aspect of the invention relates to a device for supporting bone tissue growth at a bone defect. The device comprises a tube of a semipermeable, bioabsorbable material having a first sealed end and a second, opposite sealed end. The tube comprises an interior chamber comprising bone graft granules comprising a calcium salt. The tube with the first sealed end and the second, opposite sealed end defines a deformable semipermeable pouch.

A further of the invention relates to a method for producing a device for supporting bone tissue growth at a bone defect. The method comprises applying a first seal to a tube of a semipermeable, bioabsorbable material to form an open chamber. The method also comprises filling the open chamber with a measured amount of bone graft granules or a mixture of the bone graft granules and a bone cement. The method further comprises applying a second seal to the tube to enclose the measured amount of the bone graft granules or the mixture of the bone graft granules and the bone cement within the chamber. The method additionally comprises cutting the tube to obtain a deformable semipermeable pouch made of the semipermeable, bioabsorbable material, wherein the bone graft granules comprise a calcium salt.

The device for supporting bone tissue growth at a bone defect utilizes the advantages of bone graft granules as synthetic bone graft but with reduced risk of any granule migration out from the bone defect. The deformable semipermeable pouch made of a bioabsorbable material effectively enclose the bone graft granules and thereby maintain them at the bone defect to support bone tissue growth. The device thereby reduces the risk of bone graft granules migrating away from the bone defect and into surrounding tissue.

### BRIEF DESCRIPTION OF THE DRAWINGS

The embodiments, together with further objects and advantages thereof, may best be understood by making reference to the following description taken together with the accompanying drawings, in which:

FIG. 1 is an illustration of a deformable semipermeable pouch according to an embodiment;

FIG. 2 is an illustration of a deformable semipermeable pouch according to another embodiment;

FIG. 3 is an illustration of a deformable semipermeable pouch according to a further embodiment;

FIG. 4 is an illustration of a deformable semipermeable pouch according to yet another embodiment;

FIG. 5 is an illustration of a deformable semipermeable pouch according to an embodiment;

FIG. 6 is an illustration of a deformable semipermeable pouch according to another embodiment;

FIG. 7 is an illustration of a deformable semipermeable pouch according to a further embodiment;

FIG. 8 is an illustration of a deformable semipermeable pouch according to yet another embodiment;

FIG. 9 is an illustration of bone graft granules according to an embodiment;

FIG. 10 is an illustration of a fastening screw according to an embodiment;

FIG. 11 is an illustration of a deformable semipermeable pouch according to an embodiment;

FIG. 12 schematically illustrates production of a deformable semipermeable pouch according to an embodiment; and

FIG. 13 schematically illustrates production of a deformable semipermeable pouch according to another embodiment.

### DETAILED DESCRIPTION

The present invention generally relates to devices for supporting bone tissue growth, and in particular such devices designed to be used in connection with non-confined bone defects.

Bone graft granules are common synthetic bone grafts due to their simplicity and versatility. Bone graft granules have further several advantages including biocompatibility, thereby reducing the risk of adverse immune responses, osteoconductivity by serving as a scaffold that supports new bone cell growth, controlled resorption by being engineered to resorb at a controlled rate, matching the pace of new bone formation, and reduced risk of disease transmission. One of the main advantages of bone graft granules over other types of synthetic bone grafts is their versatility. The granules can be used in various procedures, such as filling bone voids, dental implants, and spinal fusions. Their form allows for easy handling and precise application.

There is, though, a main limitation of bone graft granules and that is that they are mainly limited to so-called confined bone defects. The reason being that the bone graft granules may otherwise migrate out of the bone defect and cause negative effects to surrounding tissue or organs. In such non-bone tissue, the bone graft granules may cause inflammation and irritation, mechanical damage to the tissue, induce granuloma formation in addition to leading to impaired healing of the bone defect as the bone graft granules are not present at the intended bone defect.

The bone graft granules are thereby highly suitable for usage at confined bone defects, such as bone pockets and cavities, and other defects where the bone graft granules can be kept in place by the surrounding bone tissue. However, many bone defects are not confined, such as bone pockets or cavities, and may, for instance, be in the form of a depression in the bone surface or other type of defect that does not create a confined space at least partly confined by surrounding bone tissue. A typical example is the removal of a tumor, such as osteochondroma, osteoid osteoma, osteosarcoma, chondrosarcoma or Ewing sarcoma, from bone tissue causing a depression in the bone. Such so-called non-confined bone defects are harder to heal with bone graft granules as the bone graft granules will not be physically or mechanically kept in place in the bone defect but may unintentionally migrate or move into surrounding tissue.

Generally, non-confined bone defects are characterized by being open to surroundings. The bone defect is not enclosed by natural barriers like cortical bone or soft tissues, allowing the defect edges to remain undefined or irregular. Non-confined bone defects are harder to manage with conventional bone void fillers, such as bone graft granules, because the material can spread or migrate beyond the target area. The absence of confinement often leads to complex, irregular geometries requiring adaptable or moldable materials for bone reconstructions. Thus, without confinement, bone graft granules may flow out of the defect site during or follow application of the graft material to the bone defect.

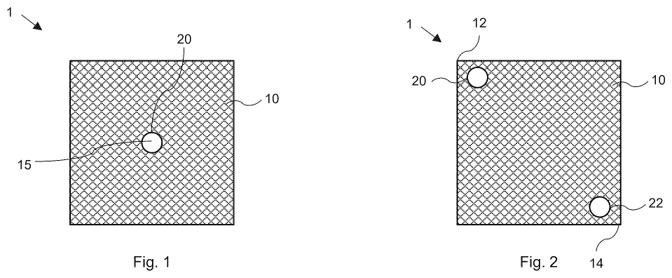

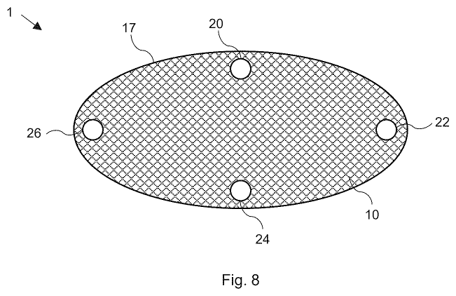

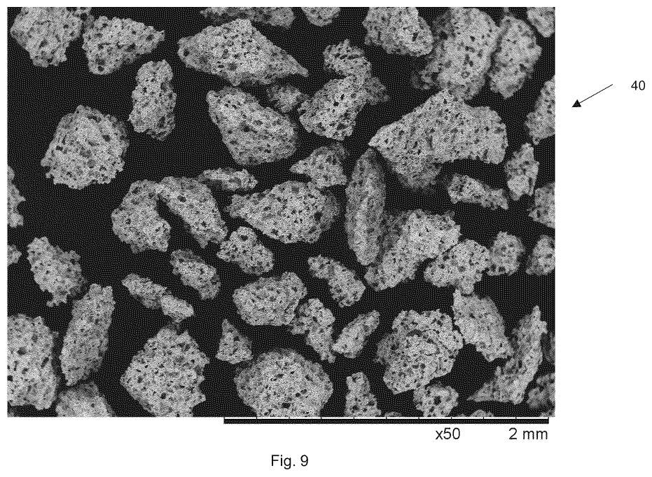

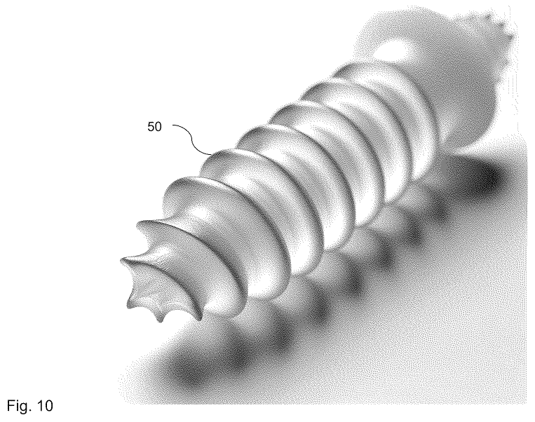

The present invention relates to a device for supporting bone tissue growth at a bone defect that utilizes the advantages of bone graft granules as synthetic bone graft but with reduced risk of any granule migration out from the bone defect. With reference to FIGS. 1-8, the device 1 comprises a deformable semipermeable pouch 10 made of a bioabsorbable material and bone graft granules 40, see FIG. 9, enclosed by the deformable semipermeable pouch 10. The bone graft granules 40 comprise a calcium salt. The deformable semipermeable pouch 10 comprises at least one through hole 20, 22, 24, 26 arranged and dimensioned for reception of a fastening screw 50, see FIG. 10, configured to screw into a bone and attach the deformable semipermeable pouch 10 at the bone.

The device 1 of the invention thereby comprises a deformable semipermeable pouch 10 made of a bioabsorbable material. This deformable semipermeable pouch 10 enclose the bone graft granules 40 therein to thereby prevent them, or at least significantly reduce the risk of them leaving the bone defect once the deformable semipermeable pouch 10 is fastened to the bone by the fastening screw 50.

The deformable semipermeable pouch 10 is made of a bioabsorbable material. This material will thereby be safely absorbed and eliminated by the body over the time. The material thereby breaks down through natural processes in the body, eventually being absorbed and excreted without the need for surgical removal. The material is further biocompatible to be compatible with body tissues and thereby minimizing the risk of any adverse reactions once implanted at a bone defect. The deformable semipermeable pouch 10 thereby provides a temporary confinement of the bone graft granules 40 present therein to maintain the bone graft granules 40 in place at the bone defect and therein support bone tissue growth. Once bone tissue has grown into the bone defect, the deformable semipermeable pouch 10 no longer serves any purpose as the bone graft granules 40 have also been absorbed or at least partly integrated with new bone growing into the bone defect and the bone graft granules 40. Thus, at that point in time, the deformable semipermeable pouch 10 can be bioabsorbed without any significant risk of any remaining bone graft granules 40 migrating out of the bone defect.

The bone graft granules 40 enclosed by the deformable semipermeable pouch 10 comprise a calcium salt. In an embodiment, the calcium salt is selected from the group consisting of hydroxyapatite (HA, Ca₅(PO₄)₃(OH)), tricalcium phosphate (TCP, Ca₃(PO₄)₂), bioactive glass, calcium sulfate dihydrate (CaSO₄·2H₂O), wollastonite (CaSiO₃), and any combination thereof.

In a particular embodiment, the calcium salt is selected from the group consisting of HA, TCP, and any combination thereof.

Hydroxyapatite (HA) is a naturally occurring mineral form of calcium apatite with the formula Ca₅(PO₄)₃(OH), often written Ca₁₀(PO₄)₆(OH)₂ to denote that the crystal unit cell comprises two entities. Up to 50% by volume and 70% by weight of human bone is a modified form of hydroxyapatite, known as bone mineral. Carbonated calcium-deficient hydroxyapatite is the main mineral, of which dental enamel and dentin are composed.

Tricalcium phosphate (TCP) is a calcium salt of phosphoric acid. TCP exists as three crystalline polymorphs α-TCP (monoclinic form), α′-TCP (hexagonal form) and β-TCP (rhombohedral form). α-TCP and β-TCP are bioresorbable with α-TCP being more soluble than β-TCP and hydrolyzes rapidly to form calcium-deficient HA. In an embodiment, TCP is in a form selected from the group consisting of α-TCP, β-TCP, and any combination thereof. In a preferred embodiment, TCP is in the form of β-TCP.

Bioactive glass is primarily composed of silicon dioxide (SiO₂), sodium oxide (Na₂O), calcium oxide (CaO), and phosphorus pentoxide (P₂O₅). Illustrative, but non-limiting, examples of bioactive glasses that could be used include 45S5 (45 wt % SiO₂, 24.5 wt % CaO, 24.5 wt % Na₂O and 6.0 wt % P₂O₅), S53P 4 (53 wt % SiO₂, 23 wt % Na₂O, 20 wt % CaO and 4 wt% P₂O₅), and 13-93 (53 wt % SiO₂, 6 wt % Na₂O, 12 wt % K₂O, 5 wt % MgO, 20 wt % CaO, 4 wt % P₂O₅).

An example of a combination of HA and TCP, preferably β-TCP, is biphasic calcium phosphate (BCP). BCP could include various weight ratios between HA and TCP, preferably β-TCP. In an embodiment, the weight ratio is selected within an interval of from HA: TCP selected within an interval of from 10%:90% weight/weight up to 90:10% weight/weight, preferably selected within an interval of from 25%:75% weight/weight up to 75%:25% weight/weight.

The bone graft granules 40 enclosed by the deformable semipermeable pouch 10 comprise, preferably are made of, HA, TCP, preferably β-TCP, and any combination thereof. The bone graft granules 40 could comprise, such as be made of, HA. In another embodiment, the bone graft granules 40 could comprise, such as be made of TCP, preferably β-TCP. In a further embodiment, the deformable semipermeable pouch 10 comprise a mixture of bone graft granules 40 comprising, preferably made of, HA and bone graft granules 40 comprising, preferably made of, TCP, preferably β-TCP.

In an embodiment, the deformable semipermeable pouch 10 comprises a mixture of the bone graft granules 40 and a bone cement.

A bone cement may be included in the deformable semipermeable pouch 10 to enable a setting reaction to occur in situ to thereby solidify the mixture of the bone graft granules 40 and the bone cement in situ in the deformable semipermeable pouch 10 once attached to the bone with the fastening screw 50.

Thus, the mixture of the bone graft granules 40 and the bone cement in the deformable semipermeable pouch 10 is, prior to polymerization of the bone cement from a paste form into a hard, solid structure, deformable and can thereby be manipulated by the surgeon during implantation of the device 1 at a bone defect. Once the device 1 has been manipulated into correct form and shape the bone cement will set, i.e., polymerize, to form a solid structure encapsulating the bone graft granules 40 in the deformable semipermeable pouch 10. Accordingly, the bone graft granules 40 will be kept in the desired three-dimensional (3D) shape and form inside the deformable semipermeable pouch 10 by the bone cement once set.

In an embodiment, the bone cement is selected from the group consisting of a calcium phosphate bone cement, a calcium sulfate bone cement, and any combination thereof.

In an embodiment, the calcium phosphate bone cement is selected from the group consisting of a brushite cement, α-TCP, tetracalcium phosphate (TTCP, Ca₄(PO₄)₂), and any combination thereof.

Brushite cement comprises primarily dicalcium phosphate dihydrate (DCPD, CaHPO₄·2H₂O). When mixed with an aqueous solution, brushite cement undergoes a setting reaction to form a solid structure. Brushite cement is bioabsorbable and osteoconductive, i.e., provides a scaffold that supports the growth of new bone cells.

α-TCP hydrolyses to calcium-deficient HA, which makes α-TCP useful in the preparation of a self-setting osteotransductive bone cement that is bioabsorbable.

TTCP is the only calcium phosphate with a Ca/P ratio greater than HA. TTCP can be used as a self-setting calcium phosphate bone cement, which forms HA under physiological conditions.

In an embodiment, the calcium sulfate bone cement is selected from the group consisting of calcium sulfate hemihydrate (CaSO₄·1/2H₂O), calcium sulfate dihydrate (CaSO₄·2H₂O), and any combination thereof.

Calcium sulfate hemihydrate can be used to prepare a fast setting, rapidly and completely bioabsorbable bone cement that is self-setting.

Calcium sulfate hemihydrate can be used to produce calcium sulfate dihydrate bioabsorbable bone cements with a setting time of 6 to 180 minutes depending on formulation and additives.

The bone cement could be in the form of a calcium phosphate bone cement, a calcium sulfate bone cement, a mixture of multiple calcium phosphate bone cements, a mixture of multiple calcium sulfate bone cements, or a mixture of at least one calcium sulfate bone cement and at least one calcium phosphate bone cement.

In an embodiment, the deformable semipermeable pouch 10 comprises the bone cement at an amount selected within an interval of from 3 up to 40% by weight of the mixture of the bone graft granules 40 and the bone cement.

In a preferred embodiment, the deformable semipermeable pouch 10 comprises the bone cement at an amount selected within an interval of from 5 up to 20% by weight of the mixture, preferably selected within an interval of from 7 up to 15% by weight of the mixture.

Thus, in an embodiment, the main component in the mixture, in terms of weight, is the bone graft granules 40 with the bone cement as the minor component and mainly used to form a solid structure that maintains the bone graft granules 40 in the desired 3D shape following implantation.

In an embodiment, the deformable semipermeable pouch 10 comprises a paste or putty comprising the bone graft granules 40. The paste or putty also comprises a polymer, preferably a water-soluble polymer, and more preferably a polymer selected from the group consisting of carboxymethyl cellulose (CMC), a poloxamer, and any combination thereof.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade names Pluronic®, Kolliphor® and Synperonic®.

Carboxymethyl cellulose (CMC) is a cellulose derivative with carboxymethyl groups (—CH₂—COOH) bound to some of the hydroxyl groups of the glucopyranose monomers that make up the cellulose backbone. It is often used in its sodium salt form, sodium carboxymethyl cellulose. The addition of carboxylic acid groups to the cellulose backbone allows carboxymethyl cellulose to be dissolved in water unlike natural cellulose.

In such an embodiment, the bone graft granules 40 can be mixed with the polymer and an aqueous solvent, preferably water, to form a paste or putty. This paste or putty comprising the bone graft granules 40 could then be enclosed in the deformable semipermeable pouch 10.

In an embodiment, the bone graft granules 40 have an average diameter selected within an interval of from 0.05 to 2 mm.

In a preferred embodiment, the bone graft granules 40 have an average diameter selected within an interval of from 0.1 up to 1 mm, preferably selected within an interval of from 0.1 up to 0.8 mm.

In an embodiment, the bone graft granules 40 are porous bone graft granules 40.

For instance, the porous bone graft granules 40 may have a porosity selected within an interval of from 5 up to 80%. In a preferred embodiment, the porous bone graft granules 40 have a porosity selected within an interval of from 5 up to 60%, preferably selected within an interval of from 5 up to 40%.

Porosity is a measure of how much of a porous bone graft granule 40 is made up of empty spaces, or pores. For a porous bone graft granule 40, porosity is defined as the ratio of the volume of the pores (void spaces) to the total volume of the porous bone graft granule 40.

In an embodiment, the porous bone graft granules 40 have an average pore diameter selected within an interval of from 1 nm up to 700 μm. In a preferred embodiment, the porous bone graft granules 40 have an average pore diameter selected within an interval of from 10 nm up to 300 μm, preferably selected within an interval of from 10 μm up to 300 μm.

Porous bone graft granules 40 with the above preferred granule dimensions, porosity and pore dimensions are in particular suitable for supporting bone tissue growth at a bone defect. FIG. 9 illustrates such porous bone graft granules 40 made of hydroxyapatite.

In an embodiment, the bioabsorbable material of the deformable bioabsorbable pouch 10 is a bioabsorbable polymer.

In an embodiment, the bioabsorbable polymer is selected from the group consisting of methyl cellulose (MC), hydroxylpropyl methylcellulose (HPMC), collagen, polyglycolide (PGA, also referred to as poly(glycolic acid)), polylactide (PLA, also referred to as poly(lactic acid)), poly-L-lactide (PLLA, also referred to as poly(L-lactic acid)), poly-p-dioxanone, poly(trimethylene carbonate), polycaprolactone (PCL), copolymers obtained by copolymerization of two or more monomers selected from glycolide, lactide, L-lactide, p-dioxanone, trimethylene carbonate and ε-caprolactone, such as poly(lactide-co-glycolide) (PGLA), and any combination thereof.

In a particular embodiment, the bioabsorbable polymer is selected from the group consisting of collagen, PGA, PLA, PLLA, PLGA, and any combination thereof.

In a preferred embodiment, the bioabsorbable polymer is selected from the group consisting of collagen, PLLA, and any combination thereof.

In another preferred embodiment, the bioabsorbable polymer is collagen.

The collagen could be in the form of type I collagen, type II collagen, type III collagen, or any combination thereof.

The collagen can be crosslinked or uncrosslinked.

In a further preferred embodiment, the bioabsorbable polymer is PLLA.

The deformable semipermeable pouch 10 comprises openings or pores and is thereby semipermeable. In an embodiment, the deformable semipermeable pouch 10 comprises openings having an average diameter selected within an interval of from 10 nm up to 1 mm. In a preferred embodiment, the deformable semipermeable pouch 10 comprises openings having an average diameter selected within an interval of from 1 μm up to 300 μm, preferably selected within an interval of from 10 μm up to 200 μm, and more preferably selected within an interval of from 50 μm up to 200 μm.

The dimensions of the openings or pores of the deformable semipermeable pouch 10 are preferably selected to support bone tissue growth at a bone defect but prevent or at least restrict the bone graft granules 40 from escaping the deformable semipermeable pouch 10 through the openings or pores.

In an embodiment, the deformable semipermeable pouch 10 has a total porosity selected within an interval from above 0 up to 70%. In a preferred embodiment, the deformable semipermeable pouch 10 has a total porosity selected within an interval from 1 up to 70%, preferably selected within an interval from 5 up to 60%, and more preferably selected within an interval from 10 up to 50%.

In an embodiment, the deformable semipermeable pouch 10 has a filling degree of the bone graft granules 40 or a mixture of the bone graft granules 40 and a bone cement selected within an interval of from 20 up to 100%. In a preferred embodiment, the deformable semipermeable pouch 10 has a filling degree selected within an interval of from 20 up to 90%, preferably selected within an interval of from 20 up to 80%.

A filling degree of 100% means that the deformable semipermeable pouch 10 is fully filled with the bone graft granules 40 or the mixture of the bone graft granules 40 and the bone cement. At such a high filling degree the deformable semipermeable pouch 10 is less deformable and is inflated like a balloon. This means that it is then very hard to deform or manipulate the deformable semipermeable pouch 10 to achieve a desired 3D shape or form of the deformable semipermeable pouch 10 and the bone graft granules 40 or the mixture of the bone graft granules and the bone cement contained therein. Hence, in some applications and in particular when there is a desire to manipulate or deform the deformable semipermeable pouch 10 in connection with implantation at a bone defect the deformable semipermeable pouch 10 preferably has a filling degree less than 100%.

Filing degree of the deformable semipermeable pouch 10 refers to the proportion of the pouch's internal volume that is occupied by the bone graft granules 40 or the mixture of the bone graft granules 40 and the bone cement.

The deformable semipermeable pouch 10 can be made according to various embodiments.

In an embodiment, a tube of the bioabsorbable material is used as material for producing the deformable semipermeable pouch 10. In such a case, the tube is preferably closed at a bottom end and then filled with a given quantity of the bone graft granules 40 or a mixture of the bone graft granules 40 and the bone cement and then closed or sealed to form the deformable semipermeable pouch 10. Such a filling process is similar to the production of snus pouches or nicotine pouches.

In another embodiment, the deformable semipermeable pouch 10 is made from two semipermeable membranes made of the bioabsorbable material. The two semipermeable membranes are then aligned on top of each other with bone graft granules 40 or the mixture of the bone graft granules 40 and the bone cement sandwiched between the semipermeable membranes. The two semipermeable membranes are then attached to each other to form the sealed deformable semipermeable pouch 10.

In a further embodiment, a single semipermeable membrane made of the bioabsorbable material is folded over itself with the bone graft granules 40 or the mixture of the bone graft granules 40 and the bone cement sandwiched between the folded parts of the semipermeable membrane. The folded semipermeable membrane is then closed or sealed to form the deformable semipermeable pouch 10.

The closure or sealing of the tube or the semipermeable membrane(s) in the above-described embodiments can be performed with, for instance, welding. For instance, the closure or sealing can be made by hot gas welding, hot plate welding, ultrasonic welding, radio frequency (RF) welding or laser welding.

The deformable semipermeable pouch 10 could be produced with various dimensions and shapes depending on the particular bone defect.

In an embodiment, the deformable semipermeable pouch 10 could have a length selected within an interval of from 10 up to 70 mm, preferably selected within an interval of from 10 up to 60 mm, and more preferably selected within an interval of from 15 up to 50 mm.

In an embodiment, the deformable semipermeable pouch 10 could have a width selected within an interval of from 5 up to 30 mm, preferably selected within an interval of from 5 up to 25 mm, and more preferably selected within an interval of from 7.5 up to 20 mm.

In an embodiment, the deformable semipermeable pouch 10 could have a wall thickness selected within an interval of from 100 μm up to 1 mm, preferably selected within an interval of from 100 up to 750 μm, and more preferably selected within an interval of from 125 up to 500 μm.

The general shape of the deformable semipermeable pouch 10 could in illustrative, but non-limiting, examples be quadratic, rectangular, cylindrical, oval, or a multi-chambered pouch 10 comprising multiple separately compartments.

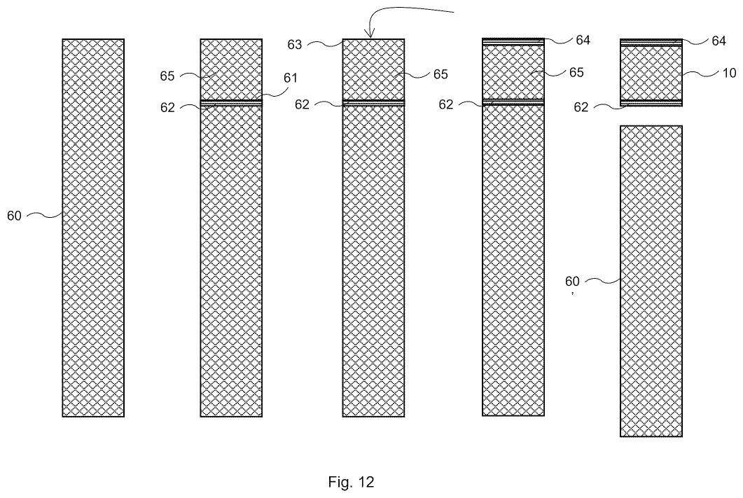

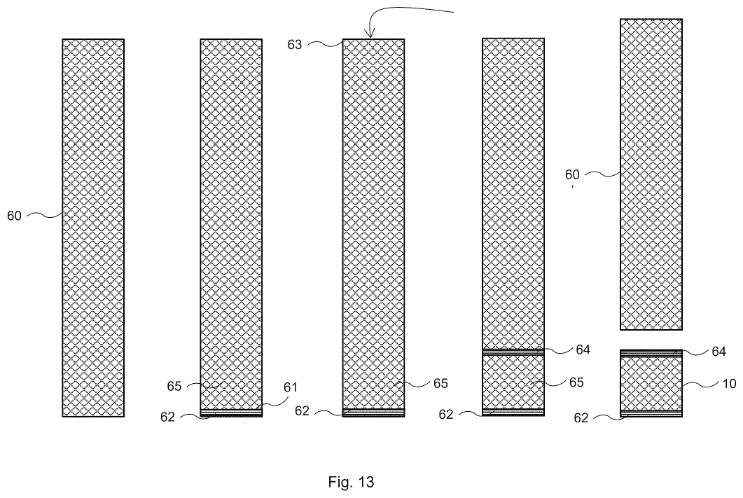

Another aspect of the invention relates to a method for producing a device 1 for supporting bone tissue growth at a bone defect, see FIGS. 12 and 13. The method comprises applying a first seal 62 to a tube 60 of a semipermeable, bioabsorbable material to form an open chamber 65. The method also comprises filling the open chamber 65 with a measured amount of bone graft granules 40 or a mixture of the bone graft granules 40 and a bone cement. The method further comprises applying a second seal 64 to the tube 60 to enclose the measured amount of the bone graft granules 40 or the mixture of the bone graft granules 40 and the bone cement within the chamber 65. The method additionally comprises cutting the tube 60 to obtain a deformable semipermeable pouch 10 made of the semipermeable, bioabsorbable material, wherein the bone graft granules 40 comprise a calcium salt.

FIG. 12 schematically illustrates an embodiment the method. An elongated tub 60 of the semipermeable, bioabsorbable material is used as starting material in this embodiment. A first seal 62, typically a first weld, is applied at the tube 60 at a position corresponding to a first or bottom end 61 of an open chamber 65 to be filled with the bone graft granules 40 or a mixture of the bone graft granules 40 and a bone cement. The bone graft granules 40 or the mixture of the bone graft granules 40 and the bone cement are then filled into the open chamber 65 through a second or top end end 63 of the open chamber 65. The chamber 65 comprising the bone graft granules 40 or the mixture of the bone graft granules 40 and the bone cement is then sealed by applying a second seal 64, typically a second weld, at the second or top end 63 of the open chamber. The sealed part of the tube 60 can then be cut to obtain the deformable semipermeable pouch 10 made of the semipermeable, bioabsorbable material. The process can then start anew to produce further deformable semipermeable pouches 10 from a remaining part 60′ of the elongated tube.

In the above-described and in FIG. 12 shown embodiment, the cutting of the elongated tube 60 is performed once both ends 61, 63 of the tube 66 have been sealed. The cutting can actually take place prior to application of the first seal 62 at the bottom end 61 of the tube 66 or at any stage following application of the first seal 62 at the bottom end 61 of the tube 66. It is, though, generally preferred to cut the elongated tube 60 to release the formed deformable semipermeable pouch 10 once both ends 61, 63 of the tube 66 have been sealed.

FIG. 13 illustrates another embodiment of the method. In this embodiment, the first seal 62 is applied at a bottom end 61 of the elongated tube 60. The measured amount of the bone graft granules 40 or the mixture of the bone graft granules 40 and the bone cement is then filled into the open chamber 65 through a top end 63 of the elongated tube 60. A second seal 64 is then applied to tube 60 to seal the chamber 65 and the bone graft granules 40 or the mixture of the bone graft granules 40 and the bone cement contained therein. The tube 60 is then cut to obtain the deformable semipermeable pouch 10 and a remaining part 60′ of the elongated tube 60.

In an embodiment, applying the first 62 comprises applying a first weld 62 to the tube 60 to form the open chamber 65 and applying the second seal 64 comprises applying a second weld 65 to the tube 60 to enclose the measured amount of the bone graft granules 40 or the mixture of the bone graft granules 40 and the bone cement within the chamber 65.

In an embodiment, welding is done by hot gas welding, hot plate welding, ultrasonic welding, radio frequency (RF) welding or laser welding.

In an embodiment, the method also comprises punching at least one through hole in the tube 60 prior to or after filling the tube with measured quantity of the bone graft granules 40 or the mixture of the bone graft granules 40 and the bone cement.

The above-described method provides a well-defined manufacture of deformable semipermeable pouches 10 containing a measured quantity of the bone graft granules 40 or the mixture of the bone graft granules 40 and the bone cement.

The deformable semipermeable pouch 10 comprises, in an embodiment, at least one through hole 20, 22, 24, 26 arranged and dimensioned for reception of a fastening screw 50 configured to screw into a bone and attach the deformable semipermeable pouch 10 at the bone. The deformable semipermeable pouch 10 can thereby be attached to bone and maintained at the bone defect by screwing the fastening screw 50 into the bone. The deformable semipermeable pouch 10 thereby comprises at least one through hole 20, 22, 24, 26 designed for reception of respective fastening screw 50. It is preferred to have the deformable semipermeable pouch 10 pre-manufactured with the at least one through hole rather than screwing one or more fastening screws 50 through the semipermeable membrane of the deformable semipermeable pouch 10. The reason for this that the fastening screw(s) 50 may then tear apart the semipermeable membrane and the deformable semipermeable pouch 10 in connection with the fastening screw(s) 50 thereby causing small openings in the deformable semipermeable pouch 10. In such a case, the bone graft granules 40 enclosed in the deformable semipermeable pouch 10 may escape through such small openings and thereby migrate into surrounding tissue.

The deformable semipermeable pouch 10 can have any shape and dimensions. For instance, the deformable semipermeable pouch 10 could be in form of a set of standard shapes, such as quadratic, rectangular and circular, and/or a set of standard sizes, such as small, medium and large. In such a case, the surgeon can select the particular standard shape and size that best match the particular bone defect. Alternatively, the deformable semipermeable pouch 10 can be made patient specific or rather defect specific to match the shape and size of a particular bone defect in a patient.

FIGS. 1-8 illustrate a number of illustrative, but non-limiting, examples of deformable semipermeable pouches 10.

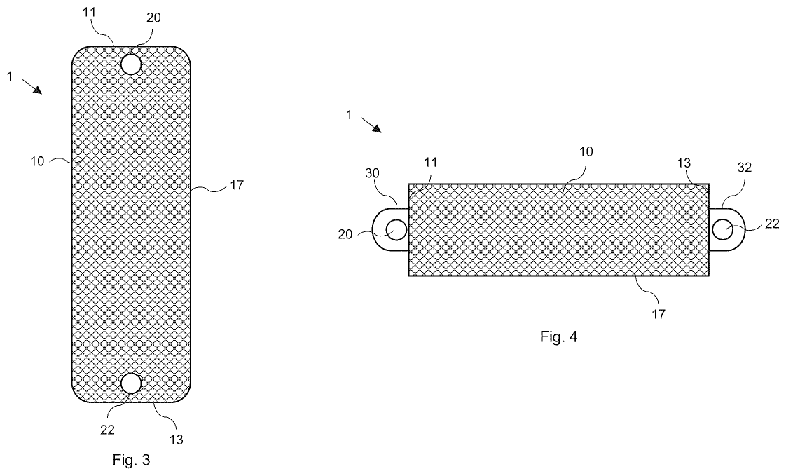

In an embodiment, the deformable semipermeable pouch 10 has a shape of a simple polygon comprising a plurality of edges 11, 13 forming a boundary or perimeter 17. In such an embodiment, the at least one through hole 20, 22 is arranged in connection with the boundary or perimeter 17. Such an embodiment is shown in FIGS. 2 and 3.

In an embodiment, the simple polygon comprises a plurality of vertices 12, 14. In such an embodiment, the at least one through hole 20, 22 is arranged in connection with at least one vertex 12, 14 of the plurality of vertices 12, 14. Such an embodiment is shown in FIG. 2.

A simple polygon is a polygon that does not intersect itself. More generally, a simple polygon is a closed curve in the Euclidean plane consisting of straight line segments, meeting end-to-end to form a polygonal chain. Two line segments meet at every endpoint, and there are no other points of intersection between the line segments. The line segments that form a polygon are called its edges or sides. An endpoint of a segment is called a vertex or a corner.

In an embodiment, the simple polygon is a simple n-gon comprising n edges 11, 13. In an embodiment, n is selected from the group consisting of 3, 4, 5, 6, 7 and 8, preferably selected from the group consisting for 3, 4, 5 and 6, and more preferably selected from the group consisting of 3, 4 and 5, such as 4.

In a preferred embodiment, the deformable semipermeable pouch 10 is thereby in the form of a triangle, a rectangle or quadrate, a pentagon, a hexagon, a heptagon, or an octagon, preferably a triangle, a rectangle or quadrate, a pentagon, or a hexagon, and more preferably a triangle, a rectangle or quadrate, or a pentagon, such as a rectangle or quadrate, see FIGS. 1-4.

In an embodiment, the deformable semipermeable pouch 10 is a deformable semipermeable rectangular or quadratic pouch comprising a first through hole 20 arranged in connection with a first corner 11 and a second through hole 22 arranged in a diagonally opposite corner 13, see FIG. 2. The deformable semipermeable pouch 10 may optionally also comprise a third through hole arranged in connection with a third corner and fourth through hole arranged in a diagonally opposite corner to thereby have a through hole 20, 22 at each corner of the rectangular or quadratic pouch.

In the case of a quadratic or rectangular pouch 10, then the sides of the quadrate or rectangle are preferably selected within an interval of from 0.5 up to 10 cm.

The deformable semipermeable pouch 10 does not necessarily have to have the at least one through hole in connection with an edge or side (FIG. 3) or in connection with a vertex or a corner (FIG. 2). One or more through holes 20 could alternatively be positioned inside the pouch 10, such as at a center 15 of the deformable semipermeable pouch 10 as shown in FIG. 1.

Such a centrally positioned through hole 20 in FIG. 1 could be combined with edge-positioned through hole(s) 20, 22 in FIG. 3 and/or vertices-positioned through hole(s) 20, 22 in FIG. 2.

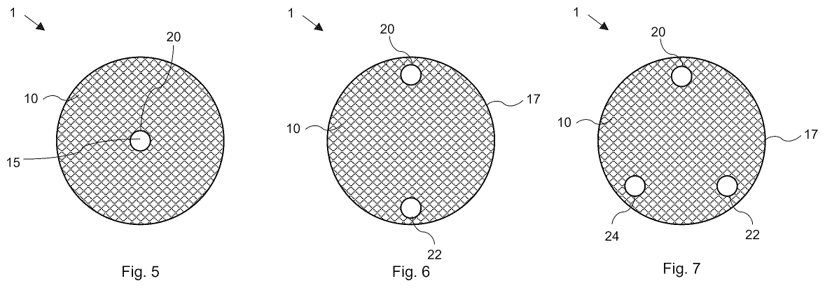

In an embodiment, see FIGS. 5-8, the deformable semipermeable pouch 10 is a deformable semipermeable circular or elliptical pouch. In such an embodiment, the at least one through hole 20 is arranged at a center 15 of the circle or at a center 15 of the ellipse, see FIG. 5.

In another embodiment, the deformable semipermeable pouch 10 is a deformable semipermeable circular or elliptical pouch. In this embodiment, the at least one through hole 20, 22, 24, 26 is arranged in connection with a border or circumference 17 of the deformable semipermeable circular or elliptical pouch, see FIGS. 6-8.

In such an embodiment and if the deformable semipermeable pouch 10 comprises multiple, i.e., at least two, through holes 20, 22, 24, 26, then the multiple through holes 20, 22, 24, 26 are preferably circumferentially distributed around the border or circumference 17 of the deformable semipermeable circular or elliptical pouch as shown in FIGS. 6-8 for the examples with two, three or four through holes 20, 22, 24, 26.

The above-described embodiments may be combined, i.e., the deformable semipermeable pouch 10 could comprise a centrally positioned through hole and one or more circumferentially positioned through holes.

In the case of a circular pouch 10, the pouch 10 preferably has a diameter selected within an interval of from 0.5 up to 5 cm.

In an embodiment, the deformable semipermeable pouch 10 comprises at least one tab 30, 32 comprising the at least one through hole 20, 22, see FIG. 4. In such an embodiment, one or more tabs 30, 32 is or are arranged at the border 17 of the deformable semipermeable pouch 10 and extend or protrude therefrom as shown in FIG. 4. In such a case, each such tab 30, 32 preferably comprise at least one through hole 20, 22.

The tab(s) 30, 32 could be made of the same bioabsorbable material as the deformable semipermeable pouch 10. However, the tab(s) 30, 30 do or does not necessarily have to be semipermeable as the bioabsorbable material of the deformable semipermeable pouch 10.

The through holes 20, 22, 24, 26 typically have a diameter selected within an interval of from 1 up to 5 mm, preferably from 3 up to 4 mm.

In an embodiment, the device 1 further comprises at least one fastening screw 50. In a preferred embodiment, the device 1 comprises at least one fastening screw 50 per through hole 20, 22, 24, 24 of the deformable semipermeable pouch 10.

The at least one fastening screw 50 is preferably made of a material selected from the group consisting of a metal or metal alloy, a ceramic, and a polymer, preferably a bioabsorbable polymer.

Illustrative, but non-limiting, examples of metals or metal alloys include stainless steel, titanium and titanium alloys, such as titanium-aluminum-vanadium alloys, e.g., Ti-6Al-4V, and cobalt-chromium alloys.

Illustrative, but non-limiting, examples of ceramics include zirconia (zirconium dioxide, ZrO₂), HA, TCP, and alumina (aluminum oxide, Al₂O₃).

Illustrative, but non-limiting, examples of bioabsorbable polymers that can be used for the bone screw can be selected from the bioabsorbable polymer materials described in the foregoing for the deformable semipermeable pouch 10.

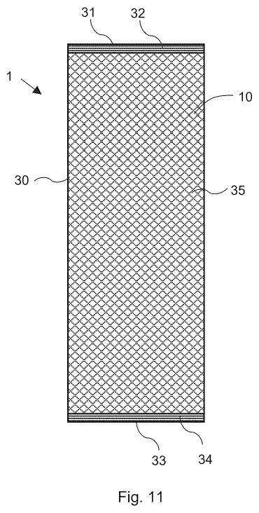

A further aspect of the invention relates to a device 1 for supporting bone tissue growth at a bone defect, see FIG. 11. The device 1 comprises a tube 30 of a semipermeable, bioabsorbable material having a first sealed end 31 and a second, opposite sealed end 33. The tube 30 comprises an interior chamber 35 comprising bone graft granules 40 comprising a calcium salt. The tube 30 with the first sealed end 31 and the second, opposite sealed end 33 defines a deformable semipermeable pouch 10.

The various embodiments described in the foregoing with reference to the deformable semipermeable pouch comprising at least one through hole also applies to this aspect of the invention except that the deformable semipermeable pouch 10 according to this aspect does not necessarily have to comprise any through hole arranged and dimensioned for reception of a fastening screw.

The tube 30 comprises a respective sealing 32, 34 at or in connection with the first and second ends 31, 33. The seals 32, 34 provided at the opposite ends 31, 33 of the tube 30 are preferably in the form of welds 32, 34. The welds 32, 34 may be formed by various welding operations, such as by hot gas welding, hot plate welding, ultrasonic welding, RF welding or laser welding.

The invention also relates to a delivery cartridge comprising at least one device 1 for supporting bone tissue growth at a bone defect. In an embodiment, the delivery cartridge comprises multiple devices 1 for supporting bone tissue growth at a bone defect.

A method of implanting a device 1 for supporting bone tissue growth comprises delivering a device 1 for supporting bone tissue growth at a bone defect using a delivery cartridge comprising at least one device 1 for supporting bone tissue growth.

### EXAMPLE 1

This Example 1 produced a collagen pouch filled with calcium phosphate granules.

Type I collagen was used as pouch material in the manufacture of an elongated pouch measuring 26 mm×12 mm with a wall thickness of 400 μm. The porosity was 30%, with an average pore size of 150 μm.

The pouch was filled with 1 mL of granules of a bioactive calcium phosphate material (a 50%/50% weight/weight mixture of HA/β-TCP granules, 0.3-1.5 mm granule size) and subsequently sealed using thermal welding. The pouch was loaded into a disposable syringe and delivered through a cannula into a metaphyseal bone defect.

### EXAMPLE 2

This Example 2 produced a highly porous PLLA pouch for controlled degradation.

A pouch was fabricated from PLLA nitting with a pore size <100 μm. The pouch dimensions were 25 mm×10 mm with a wall thickness of 250 μm.

The pouch was filled with granules of calcium phosphate granules (a 50%/50 % weight/weight mixture of HA/β-TCP granules, 0.3-1.5 mm granule size). The pouch exhibited sufficient compressive strength to withstand insertion through a delivery instrument and re-expanded after placement in a bone defect.

### EXAMPLE 3

This Example 3 produced a narrow pouch for pedicular or spinal use. This pouch is suitable for insertion between pedicles using a minimally invasive decompression (MID) instrument without risking dural injury.

A narrow cylindrical pouch was produced from a collagen/PLLA composite with a width of 11 mm and a length of 50 mm. The porosity was <40% to minimize leakage of the filler material. The wall thickness was 300 μm.

The pouch was filled with granules (see Examples 1 or 2) or a paste-like bone substitute material.

### EXAMPLE 4

This Example 4 filled pouches in a delivery cartridge to obtain a pre-filled delivery cartridge.

Three pouches (Example 1) were arranged in sequence in a delivery cartridge and configured to be expelled individually. The delivery device had an inner diameter of 6 mm and a piston mechanism. During implantation, the delivery cartridge was introduced into a bone defect and a pouch was expelled without exposing loose granules to the surrounding tissue.

### EXAMPLE 5

This Example 5 provides an automated filling of pouches using a pouch-filling machine.

A continuous roll of collagen film or PLLA film (thickness 100-800 μm) was fed into a pouch-forming and filling machine designed analogously to industrial portioning machines.

The film roll was unwound under controlled tension and was folded longitudinally to create a tubular or pocket-like structure. A continuous side seal was applied using thermal bonding, hydration or enzymatic bonding forming or ultrasound to form an open-ended tube. The open-ended tube was then indexed forward in discrete lengths corresponding to the desired pouch dimension, typically 10-40 mm.

At a filling station, the partially formed pouches passed below a granule dosing system comprising a hopper containing bone substitute granules (β-TCP, HA/β-TCP or 45S5 bioactive glass), a vibration-assisted auger or volumetric dosing cylinder, and compaction plates or vibration rails to ensure even distribution.

Each pouch received a predefined fill volume (0.25-3 mL) depending on product specification.

The automated filling of pouches in the pouch-filling machine closely mirrored the portioning action used in nicotine-pouch production, where the film forms a pocket that is precisely filled during its continuous movement.

Following filling, the open end of each pouch was sealed using the above-mentioned sealing method. Individual pouches were cut from the continuous strip using a rotary blade or a guillotine knife.

Optional embossing, pore formation, or shaping may be applied during the production step to meet specific device characteristics, including (porosity, texture, or ease of handling).

The sealed pouches were transferred to a drying or curing station, followed by, vacuum drying (if needed), and sterilization by [symbol] -irradiation, ethylene oxide sterilization, or aseptic packaging.

The final product is a ready-to-use bone-filler pouch

### EXAMPLE 6

This Example 6 produced a hydrated pouch filled with a paste-like bone substitute.

A collagen pouch with porosity 10-50% was hydrated in sterile saline to become flexible. The pouch was transferred to a wet-filling machine equipped with a peristaltic pump designed for high-viscosity biomaterials. A bioactive bone-regeneration paste was injected into the pouch at controlled pressure (10-60 kPa) until the desired fill volume was reached. The hydration allowed the pouch to conform to the paste and avoid cracking.

The pouch was sealed using a low-temperature collagen bonding technique (≤40° C.). The pouches were sterilized using [symbol] -radiation and filled into a delivery device.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A device for supporting bone tissue growth at a bone defect, the device comprising:
a deformable semipermeable pouch made of a bioabsorbable material; and
bone graft granules enclosed by the deformable semipermeable pouch, wherein the bone graft granules comprise a calcium salt, wherein
the deformable semipermeable pouch comprises at least one through arranged and dimensioned for reception of a fastening screw configured to screw into a bone and attach the deformable semipermeable pouch at the bone.

2. The device according to claim 1, wherein
the deformable semipermeable pouch has a shape of a simple polygon comprising a plurality of edges forming a boundary; and
the at least one through hole is arranged in connection with the boundary.

3. The device according to claim 2, wherein
the simple polygon comprises a plurality of vertices; and
the at least one through hole is arranged in connection with at least one vertex of the plurality of vertices.

4. The device according to claim 2, wherein the simple polygon is a simple n-gon comprising n edges, wherein n is selected from the group consisting of 3, 4, 5, 6, 7 and 8.

5. The device according to claim 2, wherein the deformable semipermeable pouch is a deformable semipermeable rectangular or quadratic pouch comprising a first through hole arranged in connection with a first corner and a second through hole arranged in a diagonally opposite corner.

6. The device according to claim 1, wherein
the deformable semipermeable pouch is a deformable semipermeable circular or elliptical pouch; and
the at least one through hole is arranged at a center of the circle or at a center of the ellipse.

7. The device according to claim 1, wherein
the deformable semipermeable pouch is a deformable semipermeable circular or elliptical pouch; and
the at least one through hole is arranged in connection with a circumference of the deformable semipermeable circular or elliptical pouch.

8. The device according to claim 1, wherein the deformable semipermeable pouch comprises at least one tab comprising the at least one through hole.

9. The device according to claim 1, further comprising at least one fastening screw.

10. The device according to claim 9, wherein the at least one fastening screw is made of a material selected from the group consisting of a metal, a metal alloy, a ceramic, and a bioabsorbable polymer.

11. The device according to claim 1, wherein the bone graft granules comprise a calcium salt selected from the group consisting of hydroxyapatite, HA, (Ca₅(PO₄)₃(OH)), tricalcium phosphate, TCP, (Ca₃(PO₄)₂), bioactive glass, calcium sulfate dihydrate (CaSO₄·2H₂O), wollastonite (CaSiO₃), and any combination thereof.

12. The device according to claim 11, wherein the bone graft granules comprise a calcium salt selected from the group consisting of, preferably selected from the group consisting of HA, TCP, and any combination thereof.

13. The device according to claim 1, wherein the deformable semipermeable pouch comprises a mixture of the bone graft granules and a bone cement.

14. The device according to claim 13, wherein the bone cement is selected from the group consisting of a calcium phosphate bone cement, a calcium sulfate bone cement, and any combination thereof.

15. The device according to claim 14, wherein the calcium phosphate bone cement is selected from the group consisting of brushite cement, alpha-tricalcium phosphate, α-TCP, tetracalcium phosphate (Ca₄(PO₄)₂), and any combination thereof.

16. The device according to claim 14, wherein the calcium sulfate bone cement is selected from the group consisting of calcium sulfate hemihydrate (CaSO₄×1/2H₂O), calcium sulfate dihydrate (CaSO₄×1/2H₂O), and any combination thereof.

17. The device according to claim 1, wherein the deformable semipermeable pouch comprises a paste or putty comprising the bone graft granules and a polymer selected from the group consisting of carboxymethyl cellulose, a poloxamer, and any combination thereof.

18. The device according to claim 1, wherein the deformable semipermeable pouch comprises the bone cement at an amount selected within an interval of from 3 up to 40% by weight of the mixture.

19. The device according to claim 1, wherein the bone graft granules have an average diameter selected within an interval of from 0.05 to 2 mm.

20. The device according to claim 19, wherein the bone graft granules have an average diameter selected within an interval of from 0.1 up to 0.8 mm.

21. The device according to claim 1, wherein the bone graft granules are porous bone graft granules.

22. The device according to claim 21, wherein the porous bone graft granules have a porosity selected within an interval of from 5 up to 80%.

23. The device according to claim 21, wherein the porous bone graft granules have an average pore diameter selected within an interval of from 1 nm up to 700 μm.

24. The device according to claim 23, wherein the porous bone graft granules have an average pore diameter selected within an interval of from 10 μm up to 300 μm.

25. The device according to any claim 1, wherein the bioabsorbable material is a bioabsorbable polymer.

26. The device according to claim 25, wherein the bioabsorbable polymer is selected from the group consisting of methyl cellulose, MC, hydroxylpropyl methylcellulose, HPMC, collagen, polyglycolide, PGA, polylactide, PLA, poly-L-lactide, PLLA, poly-p-dioxanone, poly(trimethylene carbonate), polycaprolactone, PCL, copolymers obtained by copolymerization of two or more monomers selected from glycolide, lactide, L-lactide, p-dioxanone, trimethylene carbonate and ε-caprolactone, and any combination thereof.

27. The device according to claim 26, wherein the bioabsorbable polymer is selected from the group consisting of collagen, PLLA, and any combination thereof.

28. The device according to claim 1, wherein the deformable semipermeable pouch comprises openings having an average diameter selected within an interval of from 10 nm up to 1 mm.

29. The device according to claim 1, wherein the deformable semipermeable pouch has a filling degree of the bone graft granules or a mixture of the bone graft granules and a bone cement selected within an interval of from 20 up to 80%.

30. A device for supporting bone tissue growth at a bone defect, the device comprising a tube of a semipermeable, bioabsorbable material having a first sealed end and a second, opposite sealed end, wherein
the tube comprises an interior chamber comprising bone graft granules comprising a calcium salt; and
the tube with the first sealed end and the second, opposite sealed end defines a deformable semipermeable pouch.

31. The device according to claim 30, further comprising:
a first seal at the first sealed end; and
a second seal at the second, opposite sealed end.

32. A delivery cartridge comprising at least one device for supporting bone tissue growth at a bone defect according to claim 1.

33. A method for producing a device for supporting bone tissue growth at a bone defect, the method comprising:
applying a first seal to a tube of a semipermeable, bioabsorbable material to form an open chamber;
filling the open chamber with a measured amount of bone graft granules or a mixture of the bone graft granules and a bone cement;
applying a second seal to the tube to enclose the measured amount of the bone graft granules or the mixture of the bone graft granules and the bone cement within the chamber; and
cutting the tube to obtain a deformable semipermeable pouch made of the semipermeable, bioabsorbable material, wherein the bone graft granules comprise a calcium salt.

34. The method according to claim 33, wherein
sealing the bottom end comprises sealing the bottom end of the tube by welding; and
sealing the tube comprises sealing the tube by welding.
